(19) United States
(12) Patent Application Publication
(10) Pub. No.: US 2006/0025700 A1
(43) Pub. Date: Feb. 2, 2006

(54) Method and apparatus for measuring lung temperature in real time

(76) Inventor: Joel Fallik, Yonkers, NY (US)

Correspondence Address: Ronald Abramson;Hughes Hubbard & Reed LLP, One Battery Park Plaza, New York, NY, 10004-1482, US

(21) Appl. No.: 11/174,017

(22) Filed: Jul. 1, 2005

Related U.S. Application Data
(60) Provisional application No. 60/584,651, filed on Jul. 1, 2004.

(51) Int. Cl. A61B 5/08; A61B 5/00

(52) U.S. Cl. 600/537; 600/549

(57) ABSTRACT

The invention concerns noninvasive apparatus and methods for determining the internal temperature of the lungs in real time in connection with treatments that involve heating or cooling the lungs. The method involves using a specified apparatus preferably comprising a breathing tube, temperature sensor and other elements to measure the temperature of exhaled air, and optionally combining said measurement with other measurements and parameters, in order to calculate the internal lung temperature.

### CROSS-REFERENCE

This application claims the benefit of U.S. Provisional Application Ser. No. 60/584,651, filed Jul. 1, 2004, which is incorporated herein by reference.

### BACKGROUND OF THE INVENTION

1. Field of the Invention

The present invention relates to systems enabling the controlled heating or cooling of a subject body or body portion, and more particularly to heating or cooling of a patient's lungs, and, more specifically, to noninvasive apparatus and methods for determining the internal temperature of the lungs.

2. Description of Related Art

Background information relevant to heat-based treatment is set out in a prior patent of mine in this field, Fallik, U.S. Pat. No. 5,922,013. For a further example application of this technology, see discussion under the heading “Heat-Activated Liposomes” which appears at the web page addressable at www.celsion.com/technology.htm.

It is necessary or desirable from time to time in connection with treatments and apparatus such as that described above to know the internal temperature of a patient's lungs, especially during treatment that involves applying heating or cooling to the lung area. Such treatments may be advantageously used to treat life threatening conditions, such as tuberculosis and cancer.

Preferably, it is desired to be able to monitor internal lung temperature information in real time during the course of such a treatment.

In treatments involving heating the lungs, knowing the internal lung temperature is critical. Too low a temperature reduces the therapeutic effect of the treatment. Too high a temperature can be very harmful to the patient. The object is to have a reliable enough measurement to raise the temperature as high as possible without hitting a threshold of serious injury.

The present state of the art is to use an invasive technique to measure internal lung temperature, typically, surgically inserting a temperature sensor into the lungs.

Consequently, there is a need for a noninvasive but effective mechanism for measuring the temperature of the lungs in real time.

### SUMMARY OF THE INVENTION

The present invention provides a solution to the problem described above by measuring, in alternate ways, the temperature of exhaled air, and using said measurement, in some embodiments in combination with other measurements and parameters, in calculating the internal temperature of the lungs. One aspect of the invention involves, in general, one or more of the following steps, and apparatus to carry out these steps: (1) detecting the direction of air flow in connection with the subject's breathing; (2) measuring the temperature of inflowing, inhaled air; (3) measuring the temperature of outflowing, exhaled air; (4) measuring the times of inhaling and exhaling; and (5) deriving internal lung temperature as a function or partial function of one or more of the foregoing measurements. One or more of the foregoing steps may be repeated in order to increase the accuracy of the determination.

### DETAILED DESCRIPTION

The following is a description of alternative preferred embodiments of the invention. These embodiments are illustrative only, and the invention, as defined by the claims, is by no means limited to particular examples shown. For example, certain preferred embodiments are described in relation to an implementation with specific fasteners, sensors and tubing, but it should be appreciated that the disclosure that follows was intended to enable those skilled in the art readily to apply the teachings set forth to other commonly available hardware and electronics. The specific features of any particular embodiment should not be understood as limiting the scope of what may be claimed.

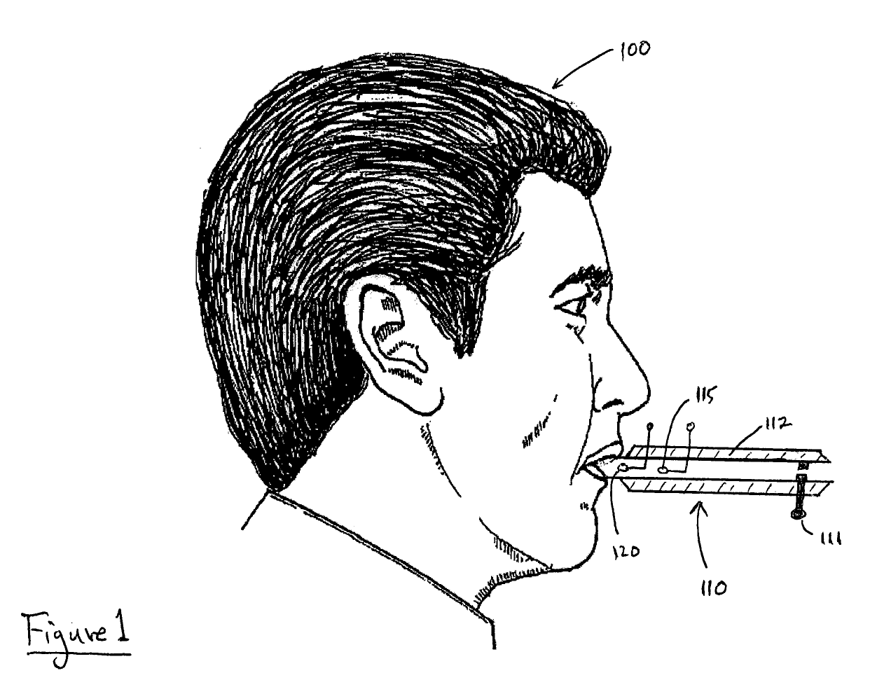

Referring to FIG. 1, in one preferred embodiment, a breathing tube, 110, preferably insulated (with insulation, 112), as shown. The breathing tube is inserted in, and held by, the mouth of subject 100. Optionally, the breathing tube 110 may be installed in a face mask (not shown). Direction sensor 115 senses the direction of the flow of air through the breathing tube. Direction sensor 115 could be, for example, a propeller, pressure gauge, diaphragm or the like. Alternatively, a strap across the patient's chest (not shown) could be employed as a direction sensor, sensing expansion or contraction of the chest and an indication of whether the patient is inhaling or exhaling. Preferably, breathing tube 110 has an adjustable air exit 111 so as to regulate the air flow resistance of the tube. Temperature, direction and other measurements are digitally recorded against a time base, so as to maintain a time line of relevant measurements.

Temperature sensor 120 proximate the patient's mouth and within the air flow into and out of said breathing tube, measures the temperature of the air flowing over the sensor. Alternately, a plurality of temperature sensors could be used.

One method of operation involves having the subject breathe through the apparatus, inhaling only through the nose and exhaling only through the mouth. Prior to measurement, the subject should breathe deeply through the apparatus to equilibrate it thermally (or at least reach an approximate thermal steady state). The flow resistance should preferably be adjusted so that a positive pressure is maintained during most of the breathing cycle, to avoid mixing with outside air but at the same time maintain a steady flow of new air with every exhaled breath

Other inputs could include ambient temperature and/or humidity, altitude, barometric pressure, air flow velocity, and the size, weight and/or lung capacity of the patent of the patient.

Optionally, a nose clip could be used to force mouth breathing during the measurement, although in general it may be more advantageous to have the subject inhale nasally and exhale through the mouth. Another option would be to insert the breathing tube onto the nose of the patient so as to isolate exhaled air from ambient air in the vicinity of the temperature sensor. The best approach may vary depending on the condition of the subject, and the subject's ability to reliably follow a set breathing pattern during the procedure.

The internal lung temperature can be approximated by the measured temperature of exhaled air. This could be a measurement by temperature sensor 120 at any time, but preferably would be a measurement when the subject is exhaling, as indicated by direction sensor 115 (or alternate means, such as a chest strap).

Generally, actual internal lung temperature will be higher than the temperature measured at sensor 120. At normal ambient temperatures (20-25 degrees Celsius) the inhaled air will not in general heat up to the actual internal lung temperature. In addition, if the lungs are being heated, the exhaled air will have the opportunity to lose temperature on the way out of the breathing tract. Further cooling may take place in the measuring apparatus, as a result of surface conduction and mixing with non-exhaled air. The difference will be a function of at least the following:
rate of breathing (slower tends toward higher exhalent temperatures)
volume of breathing (deeper breathing tends toward higher exhalant temperatures)
separation of inhaled air from exhaled air
ambient temperature
humidity
barometric pressure
altitude
size, weight and/or lung capacity of the subject

To a first approximation, exhaled air measured under thermal equilibrium conditions in an insulated breathing tube in the mouth which is only exhaled through and flow-restricted so as to maintain a positive pressure, with an ambient temperature of about 21 degrees Celsius at normal barometric pressure at sea level and about 75% humidity is about 1.5-2.0 degrees Celsius lower than internal lung temperature. A further temperature difference increment to provide a margin of safety may be optionally added to compensate for the small uncertainty in this measurement.

The invention is not limited to human use and may be used with animals.

The effect of the factors given above may be refined by further experimentation, if necessary.

It is evident that the embodiments described herein accomplish the stated objects of the invention. While the presently preferred embodiments have been described in detail, it will be apparent to those skilled in the art that the principles of the invention are realizable by other devices, systems and methods without departing from the scope and spirit of the invention, as defined in the following claims.

I claim:

1. A method for determining the temperature of a subject's lungs in real time, comprising the step of measuring the temperature of air exhaled by the subject.

2. The method of claim 1, further comprising the steps of:
a) having the subject inhale through the nose and exhale through the mouth
b) inserting a breathing tube in the subject's mouth, said breathing tube having a temperature sensor in the inside thereof proximate the mouth opening of the tube, thermal insulation, and an air exit of adjustable cross-section
c) having the subject breath through the tube in order to warm it up to an approximate steady state temperature
d) adjusting said exit cross-section so as to maintain a positive pressure in the tube during most of the breathing cycle
e) taking a series of timed temperature measurements with said sensor, and recording said measurements in digital, machine-readable form.

3. The method of claim 2, wherein the apparatus employed, as recited in claim 2, has in addition a sensor for detecting whether the subject is inhaling or exhaling, and temperature measurements are limited to the period during which the subject is exhaling.
